(19) United States
(12) Patent Application Publication
(10) Pub. No.: US 2026/0232314 A1
(43) Pub. Date: Aug. 13, 2026

(54) SURGICAL INSTRUMENT WITH LOCKOUT MECHANISM

(71) Applicant: INTUITIVE SURGICAL OPERATIONS, INC., Sunnyvale, CA (US)

(72) Inventors: Atal C. Patel, Mission Viejo, CA (US); Matthew Wixey, San Jose, CA (US)

(73) Assignee: INTUITIVE SURGICAL OPERATIONS, INC., Sunnyvale, CA (US)

(21) Appl. No.: 19/638,266

(22) Filed: Apr. 3, 2026

Related U.S. Application Data
(63) Continuation of application No. 18/747,654, filed on Jun. 19, 2024, now Pat. No. 12,648,774.
Application No. 18/747,654 is a continuation of application No. 17/890,362, filed on Aug. 18, 2022, now Pat. No. 12,137,903.
Application No. 17/890,362 is a continuation of application No. 16/969,483, filed on Feb. 26, 2019, now Pat. No. 11,439,390.
(60) Provisional application No. 62/635,348, filed on Feb. 26, 2018.

(51) Int. Cl. A61B 17/068; A61B 17/072

(52) U.S. Cl. CPC A61B 17/0686; A61B 17/072; A61B 2017/07264; A61B 2017/07271; A61B 2017/07278; A61B 2017/07285

(57) ABSTRACT

Locking assemblies for surgical clamping and cutting instruments include a locking member and a switch. A drive member may be configured to releasably engage a knife and/or a shuttle of the surgical instrument for translating the knife and/or shuttle in a distal direction through a firing stroke. The locking member is movable from a first position permitting distal translation of the drive member through the firing stroke, and a second position inhibiting distal translation of the drive member through the firing stroke. A switch, when proximally positioned, releasably engages the locking member to maintain the locking member in the first position. The switch disengages from the locking member when the switch is moved to a distal position.

### CROSS-REFERENCE TO RELATED APPLICATIONS

This application is a continuation of U.S. Patent Application Serial No. 18/747,654 filed June 19, 2024, which is a continuation of U.S. Patent Application Serial No. 17/890,362 filed August 18, 2022, Issued as U.S. Patent No. 12,137,903 on November 12, 2024, which is a continuation of U.S. Patent Application Serial No. 16/969,483 filed August 12, 2020, Issued as U.S. Patent No. 11,439,390 on September 13, 2022, which is a National Stage of International Application No. PCT/US19/19501 filed February 26, 2019 which claims benefit of U.S. Provisional Application No. 62/635,348 filed February 26, 2018, the entirety of which is herein incorporated by reference.

### TECHNICAL FIELD

The present disclosure relates to surgical stapling instruments having a locking mechanism to prevent actuation of a knife when there is a spent or previously fired cartridge in place.

### BACKGROUND

Surgical clamping and cutting instruments, such as, for example, surgical stapling instruments, may include an end effector having opposing jaws that clamp tissue and a knife that cuts the clamped tissue. It is often advantageous for an end effector of a surgical stapling instrument to be reusable. To that end, staple cartridges can be fitted into one jaw of the end effector prior to each use of the surgical stapling instrument.

It is desirable to prevent firing of a surgical stapling instrument while a spent cartridge remains in place on the jaw. Thus, a need exists for effective mechanisms to prevent firing of a surgical stapling instrument while a spent staple cartridge is in place in the end effector of the surgical stapling instrument.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present disclosure relates to surgical stapling instruments having a locking mechanism. Surgical stapling instruments described herein employ a locking mechanism to prevent actuation of a knife when there is a spent or previously fired cartridge in place.

In one aspect, a surgical stapling instrument in accordance with this disclosure includes an anvil jaw assembly and a staple jaw assembly. The staple jaw assembly includes a channel configured to receive a staple cartridge, the staple cartridge including a shuttle. The channel includes two side walls, a bottom wall, and a cam surface adjacent the bottom wall. The surgical stapling instrument further includes a drive member configured to releasably engage and translate the shuttle in a distal direction through a staple firing stroke. The locking member is pivotable between a first position permitting distal translation of the drive member, and a second position preventing distal translation of the drive member. The drive member further includes a footer configured to engage the cam surface and simultaneously pivot the channel, the shuttle, and the locking member towards the anvil assembly.

In another aspect, a surgical stapling instrument in accordance with this disclosure includes an anvil jaw assembly, a drive member configured to translate in a distal direction through a staple firing stroke, and a locking member pivotable between a first position permitting distal translation of the drive member, and a second position preventing distal translation of the drive member. The staple jaw assembly further includes a channel configured to receive a staple cartridge, the staple cartridge including a shuttle. The channel includes two sidewalls, each sidewall having a guide structure configured to direct a proximal ramped surface of the shuttle into contact with a distal ramped surface of the locking member to urge a portion of the locking member onto a shelf of the shuttle to maintain the locking member in the first position.

In another aspect, a surgical stapling instrument in accordance with this disclosure includes an anvil jaw assembly and a staple jaw assembly. The staple jaw assembly includes a channel configured to receive a staple cartridge, the staple cartridge including a shuttle. The channel includes two side walls and a bottom wall having a pair of openings. The surgical stapling instrument further includes a drive member configured to releasably engage and translate the shuttle in a distal direction through a staple firing stroke. A locking member is pivotable between a first position permitting distal translation of the drive member, and a second position preventing distal translation of the drive member. The locking member includes a pair of feet configured to pass through respective openings in the bottom wall of the channel. The pair of feet are movable from a first position preventing removal of the feet from the openings, to a second position allowing removal of the feet from the openings.

In another aspect a surgical instrument is provided, including an anvil assembly and a staple jaw assembly including a channel defining a longitudinal axis and configured to receive a staple cartridge. The staple cartridge includes a shuttle. A drive member is configured to releasably engage and translate the shuttle in a distal direction through a staple firing stroke. The surgical stapling instrument further includes a locking member pivotable between a first position permitting distal translation of the drive member, and a second position preventing distal translation of the drive member. The locking member includes a pair of distally extending arms, each arm having a proximally facing chamfered surface configured to engage and align the drive member along the longitudinal axis of the channel as the drive member translates distally between the arms of the locking member.

### BRIEF DESCRIPTION OF THE DRAWINGS

The above and other aspects, features, and advantages of the present surgical stapling instruments having a locking mechanism will become more apparent in light of the following detailed description when taken in conjunction with the accompanying drawings in which:

FIG. 1A is a perspective view of an illustrative surgical stapling instrument including a stapling jaw assembly, and an anvil jaw assembly;

FIG. 1B is an exploded view of an illustrative end effector of a surgical stapling instrument;

FIG. 2A is a top perspective view of the channel and locking assembly of the end effector of FIG. 1B;

FIG. 2B depicts a partial perspective view of the proximal end of the channel and locking member of FIG. 2A;

FIG. 3 is a partial cross-sectional view of the end effector of FIG. 1B in an open position;

FIG. 4 is a partial cross-sectional side view of the end effector of the end effector of FIG. 1B in a partially closed position;

FIG. 5 is a partial side, cross-sectional view of the end effector of the end effector of FIG. 1B in a substantially closed position;

FIG. 6A is a side cross-sectional view of the end effector of FIG. 1B in a closed position;

FIG. 6B is a partial rear perspective view of the end effector in the closed position with a staple cartridge in place;

FIG. 7 is a partial side, cross-sectional view of the end effector of FIG. 1B showing the locking member in the locking position;

FIG. 8 is a partial side, cross-sectional view of the end effector of FIG. 1B showing the drive member and shuttle advanced distally;

FIG. 9 is a partial side, cross-sectional view of the end effector of FIG. 1B showing the drive member and shuttle advanced to the distal-most position;

FIG. 10A is a partial cross-sectional view showing the drive member interacting with the locking member upon retraction of the drive member;

FIG. 10B is a partial perspective view of FIG. 10A;

FIG. 11 is a partial cross-sectional view of the end effector of FIG. 1B illustrating a ramped portion of the drive member riding under the locking member during retraction;

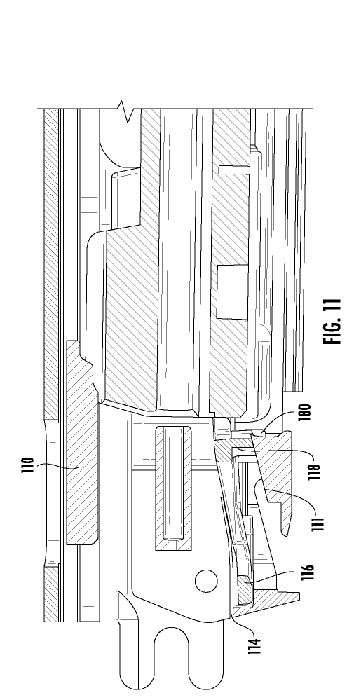

FIG. 12 is a partial cross-sectional view of the end effector of FIG. 1B illustrating the drive member retracted sufficiently to allow the locking member to move to the locking position;

FIG. 13 is a partial cross-sectional view of the end effector of FIG. 1B illustrating a reload-specific shuttle engaging a distal portion of a locking member;

FIG. 14 is a partial cross-sectional view of the end effector of FIG. 1B illustrating another embodiment of a reload-specific shuttle engaging a distal portion of the locking member;

FIG. 15 is a partial cross-sectional view of the end effector of FIG. 1B illustrating a third embodiment of a reload-specific shuttle engaging a distal portion of the locking member;

FIG. 16 is a partial cross-sectional view of the end effector of FIG. 1B illustrating a fourth embodiment of a reload-specific shuttle engaging a distal portion of the locking member;

FIG. 17 is a perspective view of the channel of an embodiment of a staple jaw assembly having a guide structure present on the sidewalls of the channel;

FIG. 18 is a close-up of the guide structure of FIG. 17;

FIG. 19 is a partial perspective view of a reload being installed into the channel of a surgical stapling instrument in accordance with the embodiment of FIG. 18, with one of the sidewalls of the channel removed;

FIG. 20 is a partial perspective view of the distal end of a channel and a locking member in accordance with the embodiment of FIG. 2 illustrating a guard wall of the locking member bottom;

FIG. 21 is a bottom view of the channel of FIG. 20 showing the feet of the locking member positioned through openings in the bottom wall of the channel;

FIG. 22 is a bottom perspective view of a locking member in accordance with the embodiment of FIG. 21;

FIG. 23 is a top perspective view of a locking member in accordance with the embodiment of FIG. 21;

FIGS. 24 A, B, and FIG. C are a front, side, and perspective view, respectively, of a drive member of a surgical stapling instrument configured to engage a locking member in accordance with the embodiment of FIGS. 22-23;

FIG. 25 is a partial side view of the drive member in accordance with the embodiment of FIG. 24 engaging chamfered surfaces of the locking member while the locking member is in the unlocked position; and

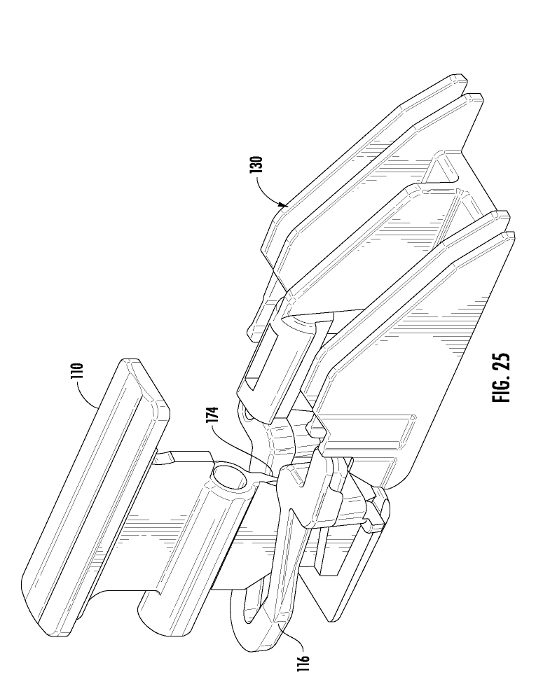

FIG. 26 is a partial top view of FIG. 25.

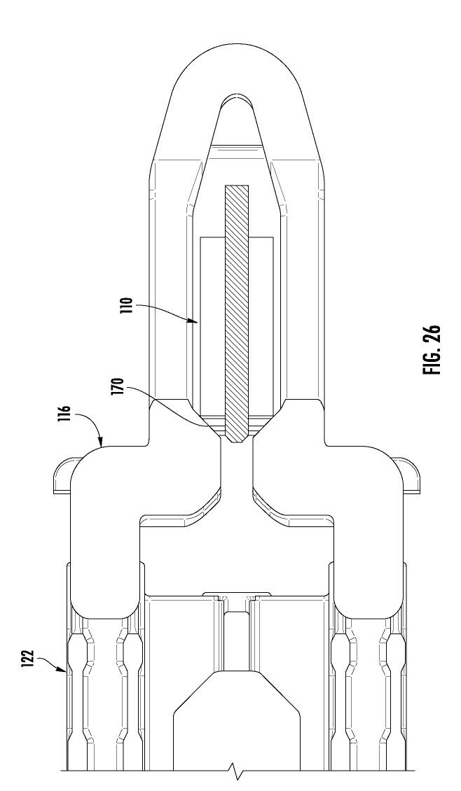

### DETAILED DESCRIPTION

Particular embodiments of the present surgical stapling instruments are described hereinbelow with reference to the accompanying drawings; however, it is to be understood that the disclosed embodiments are merely exemplary of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Well-known functions or constructions are not described in detail to avoid obscuring the present disclosure in unnecessary detail.

The present disclosure relates to surgical stapling instruments including a locking member and a drive member configured to engage at least one of a knife or a shuttle of a surgical stapling instrument and to translate the same in a distal direction through a staple firing stroke. Contact between the drive member and the knife and/or shuttle is releasable in that once the knife and/or shuttle are translated by the drive member in the distal direction through a staple firing stroke, the knife and/or shuttle disengages from the drive member, remains at a distal portion of the stapling instrument, and is not translated in a proximal direction by the drive member. The locking member is movable from a first position permitting distal translation of the drive member through the staple-firing stroke, and a second position inhibiting distal translation of the drive member through the staple firing stroke. A spring is configured to bias the locking member toward the second position. When the shuttle is in the proximal position, the shuttle releasably maintains the locking member in the first position. When the shuttle advances distally, the shuttle disengages from the locking member thereby allowing the locking member to move to the second position.

While the following disclosure is presented with respect to a linear surgical stapler where staples are sequentially fired, it should be understood that the present locking assemblies may be readily adapted for use in any type of surgical clamping and cutting instruments, whether or not the surgical clamping and cutting instrument applies a fastener. The surgical clamping and cutting instrument may be a minimally invasive (e.g., laparoscopic) instrument or an instrument used for open surgery.

Additionally, the present locking assemblies may be readily adapted for use in surgical instruments that are activated using any technique within the purview of those skilled in the art, such as, for example, manually activated surgical instruments, powered surgical instruments (e.g., electro-mechanically powered instruments), robotic surgical instruments, and the like.

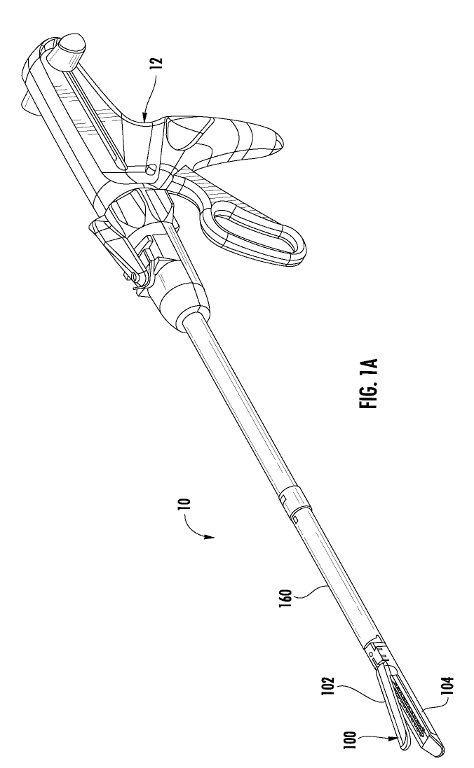

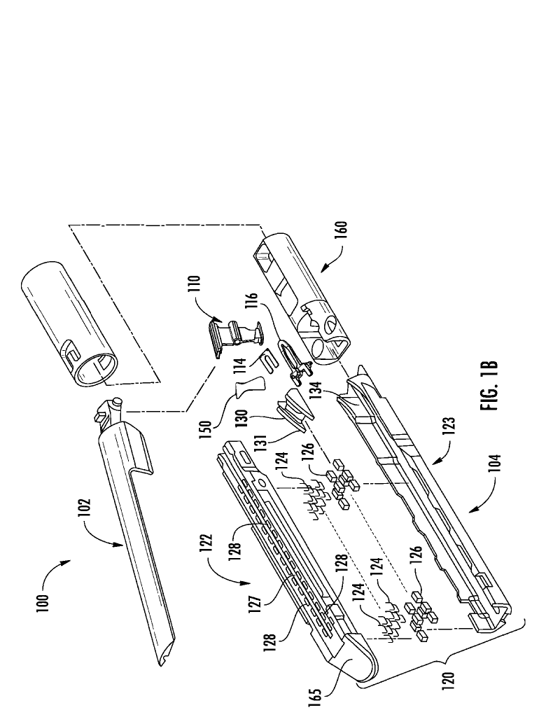

FIG. 1A is a perspective view of an illustrative surgical stapling instrument 10 capable of utilizing a locking assembly in accordance with the present disclosure. Surgical stapling instrument 10 includes a handle assembly 12, and an end effector 100 including an anvil jaw assembly 102 and a staple jaw assembly 104 mounted on an elongated shaft 160 of the surgical stapling instrument 10.

FIG. 1B shows anvil jaw assembly 102, including an anvil having staple forming pockets (not shown) supported thereon, and staple jaw assembly 104. Staple jaw assembly 104 and anvil jaw assembly 102 are configured to move from an open position to a closed position. In the open position, a fresh stapling cartridge (sometimes referred to as a reload) can be loaded into staple jaw assembly 104, a spent staple cartridge removed from staple jaw assembly 104, and tissue may be positioned between the jaw assemblies 102, 104. In the closed position, jaw assemblies 102, 104 cooperate to close upon and clamp tissue such that cartridge 122 and the anvil (not shown) are in close cooperative alignment. In the embodiment shown in FIGS. 1A and 1B, staple jaw assembly 104 is stationary and anvil jaw assembly 102 pivots to the open position. In other embodiments it is contemplated that the jaw assembly containing the anvil is stationary and the jaw assembly containing the staple cartridge pivots to the open position. As those skilled in the art reading this disclosure will appreciate, in yet other embodiments both the anvil jaw assembly and the staple jaw assembly may pivot.

With continued reference to FIG. 1B, staple jaw assembly 104 includes a staple cartridge 122 supported in a channel 134 on jaw 123. Cartridge 122 includes a plurality of staples 124 that are supported on corresponding staple drivers 126 provided within respective staple apertures 128 formed in cartridge 122. Cartridge 122 also includes a shuttle 130 having an inclined distal portion 131 that, upon distal movement, sequentially acts on staple drivers 126, camming them upwardly thereby moving staples 124 into deforming contact with the anvil (not shown). Cartridge 122 also includes a knife 150 configured to translate distally through a channel 127 in cartridge 122 and to sever clamped, stapled tissue.

FIG. 1B further shows a drive member 110 movably supported on the surgical stapling instrument such that it may pass distally through cartridge 122 and staple jaw assembly 104 when the surgical stapling instrument is fired (e.g., actuated). Also shown in FIG. 1B is the locking assembly including locking member 116 and spring 114.

With a fresh reload installed, drive member 110 is in a proximal position where it has not yet engaged knife 150 or shuttle 130. Drive member 110 may be any structure capable of pushing at least one of a shuttle or a knife of a surgical stapling instrument with the necessary force to effectively sever or staple human tissue. Drive member 110 may be an I-beam, an E-beam, or any other type of drive member capable of performing similar functions.

For a more detailed description of illustrative end effectors, reference may be made to U.S. Patent Nos. 6,669,073 and 8,800,841, the entire contents of which are incorporated herein by this reference. It should of course, be understood that end effector shown in FIGS. 1A and 1B is merely illustrative, and that other end effectors may be employed, including but not limited to the end effectors shown in WO2014/106275, the entire contents of which are incorporated herein by this reference.

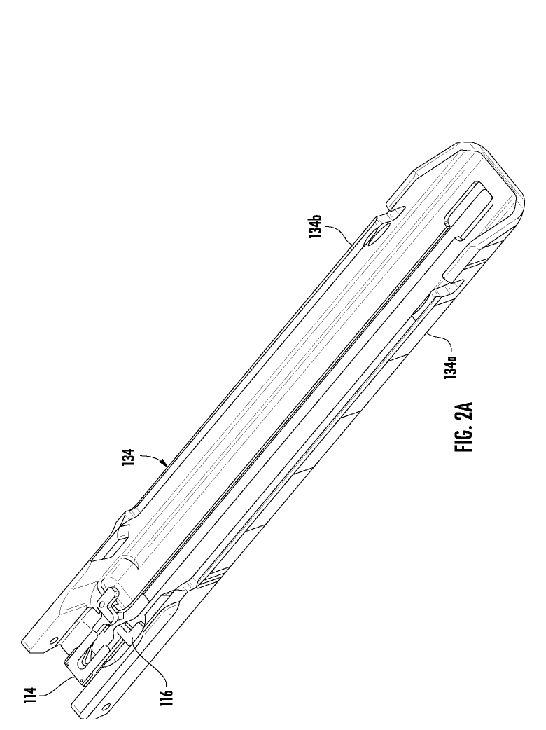

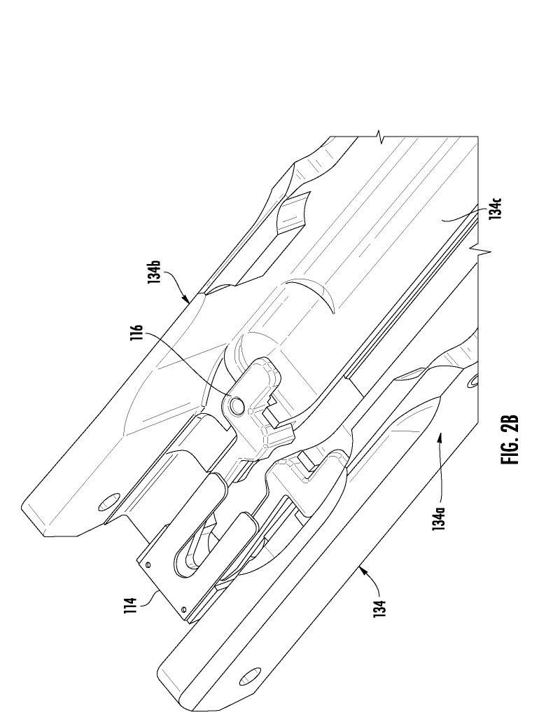

FIGS. 2A and 2B show channel 134, spring 114, and locking member 116 of the end effector of a surgical stapling instrument in accordance with an embodiment of the present disclosure. Channel 134 includes two side walls 134a, b and a bottom wall 134c.

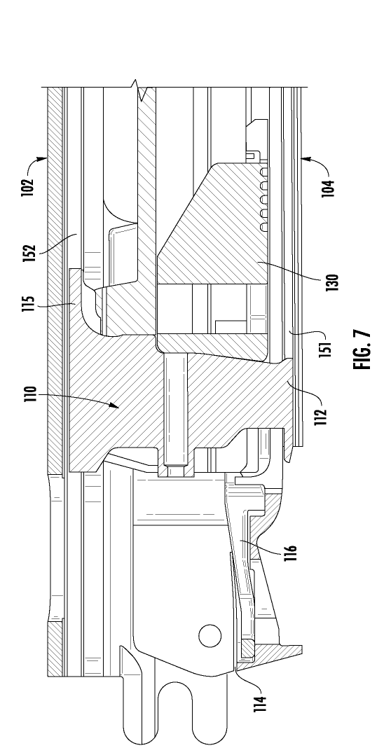

In an illustrative embodiment of a surgical stapling instrument in accordance with this disclosure, locking member 116 is positioned in the proximal end of channel 134. Spring 114 serves to bias locking member 116 into a second position preventing distal movement of a drive member 110 (See FIG. 7).

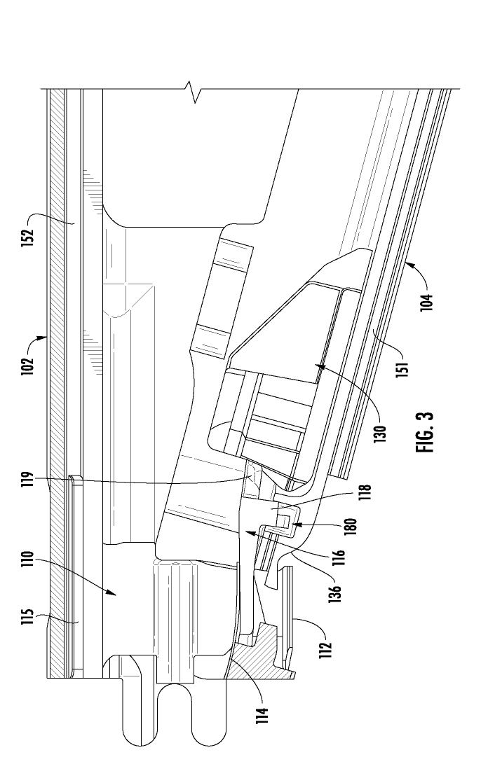

FIGS. 3-5 illustrate the interaction of drive member 110 with a cam surface 136 of the channel 134 to simultaneously pivot channel 134 and its contents.

As seen in FIG. 3, an illustrative surgical stapling instrument may include drive member 110 having a footer 112, spring 114, channel 134 having a cam surface 136, and locking member 116. Also shown in FIG. 3 is shuttle 130, which is contained within staple cartridge 122, the structure of which is not shown in this view.

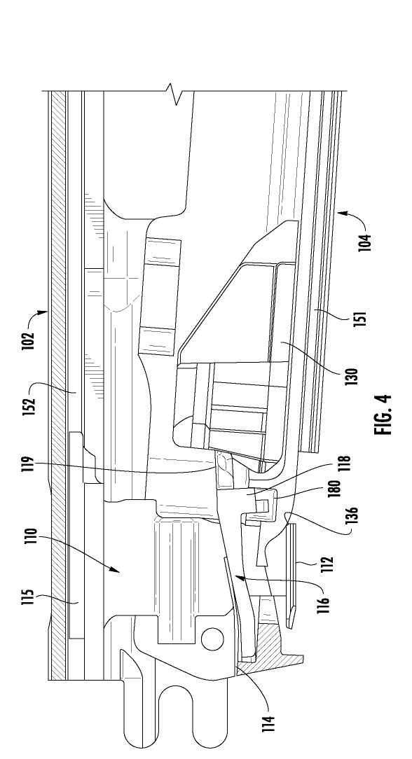

As a user manipulates handle assembly 12 to close the jaws of the surgical stapling instrument, drive member 110 moves distally, causing a footer 112 of the drive member to come into contact with cam surface 136 of channel 134. As shown in FIGS. 4-6, as drive member 110 rides along cam surface 136, simultaneous pivoting of the channel 134, the shuttle 130, and the locking member 116 towards the anvil jaw assembly is achieved.

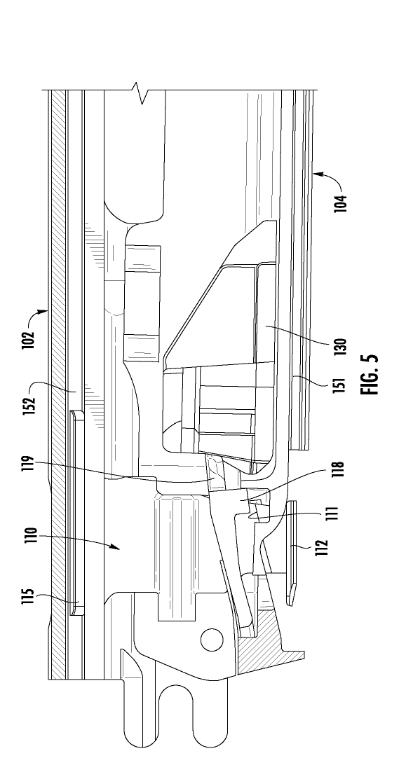

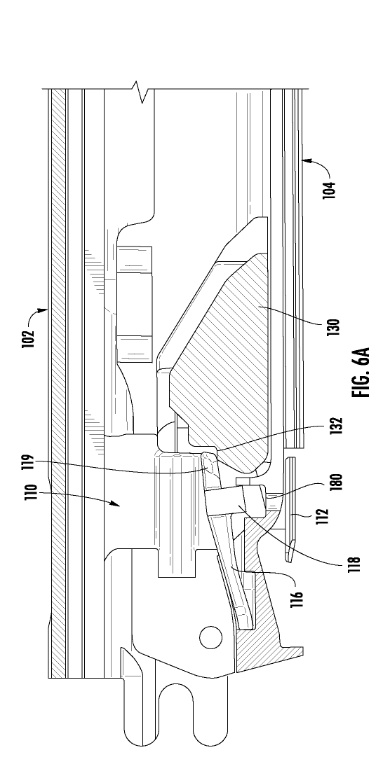

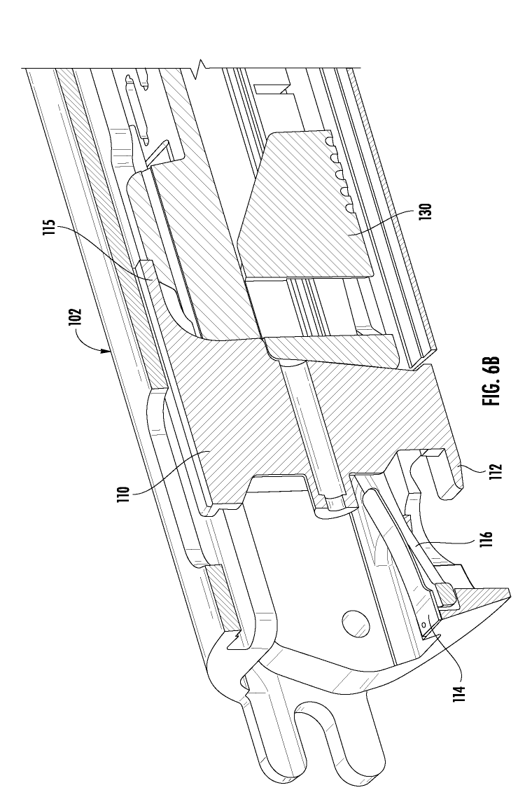

The timing of this pivoting allows for a ramped portion 111 of the drive member to clear underneath an engagement portion 118 of locking member 116, as shown in FIG. 5. During pivoting, distal portion 119 of locking member 116 is resting on shelf 132 of shuttle 130, (See FIG. 6A) and is therefore in a first position allowing for distal translation of drive member 110. In this first position, engagement portion 118 of the locking member 116 is positioned above a slot 180 in channel 134.

FIGS. 6-9 sequentially illustrate the actuation of an illustrative surgical stapling instrument in accordance with the present disclosure.

In FIGS. 6A and B, the actuation stroke has begun and drive member 110 has advanced distally to contact shuttle 130, but shuttle 130 has not yet moved. Lower ramped portion 111 is still able to pass underneath locking member 116, as the locking member continues to rest on the shelf 132 of shuttle 130 and is in the first unlocked position.

In FIG. 7, drive member 110 continues to travel distally down channel 134, translating shuttle 130 in a distal direction. Once shuttle 130 starts to translate distally, locking member 116 falls off of shelf 132 of shuttle 130 causing engagement portion 118 of locking member 116 to move to a second locking position by falling into and engaging slot 180, thereby enabling the locking mechanism. Spring 114 is configured to bias engagement portion 118 of locking member 116 toward slot 180 to keep the locking mechanism enabled.

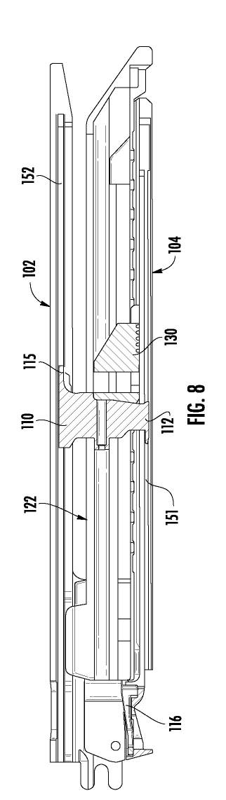

As drive member 110 continues to move distally, as shown in FIG. 8, footer 112 rides within slot 151 formed in the bottom wall 134c of channel 134 and top member 115 of drive member 110 rides within slot 152 of anvil assembly 102 to maintain the jaw assemblies 102, 104 spaced a constant distance from each other.

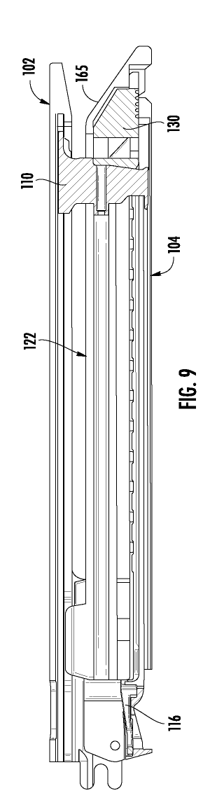

Ultimately, as shown in FIG. 9, drive member 110 translates distally through a complete firing stroke during which stapling and severing of tissue have occurred. Drive member 110 can then be retracted, leaving shuttle 130 parked at a position in a distal portion of cartridge 122. In embodiments, such as the embodiment illustrated in FIG. 9 shuttle 130 may be unable to move proximally towards the home position due to friction with cartridge 122. In embodiments, a knife may be integrally formed with drive member 110. In embodiments, a separate knife 150 (See FIG. 1B) may be translated distally by drive member 110 and may be parked in a predetermined position in a distally located garage 165, the garage 165 including lateral surfaces that face the cutting edge of knife 150.

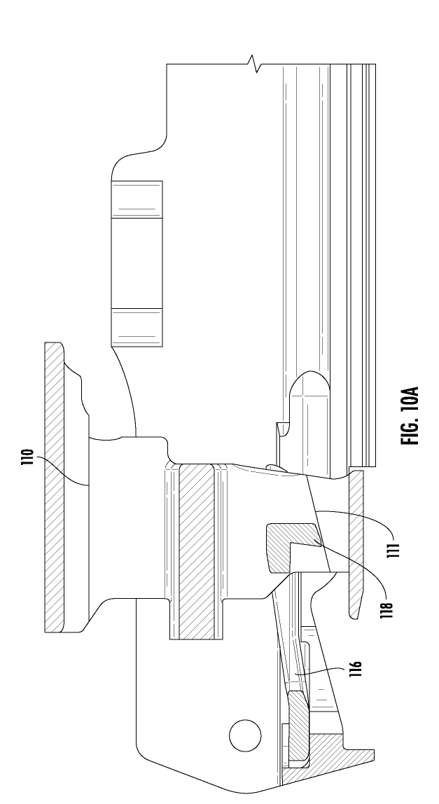

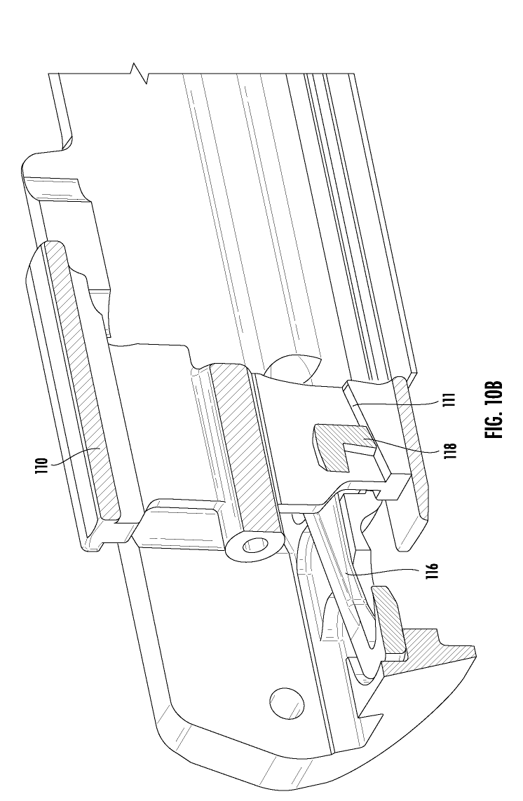

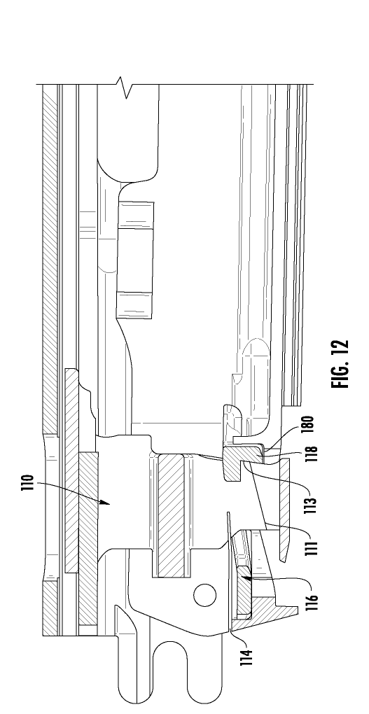

FIGS. 10A and FIG. B and 11 illustrate retraction of drive member 110 after a firing stroke has been completed. As drive member 110 is retracted to the proximal end of the end effector, lower ramped portion 111 of drive member 110 contacts and rides under engagement portion 118 of locking member 116, causing locking member 116 to be pushed upwards against the bias of spring 114. Once locking member 116 has passed over the entirety of lower ramped portion 111 of drive member 110 as seen in FIG. 12, the bias of spring 114 again forces engagement portion 118 of locking member 116 to fall back into and engage slot 180, thereby engaging the locking mechanism.

FIG. 12 shows an illustrative embodiment of a surgical stapling instrument that has been locked out. If a user attempts to actuate a surgical stapling instrument in this position, a distal face 113, located on the distal end of lower ramped portion 111 of drive member 110, will abut the proximal side of engagement portion 118 of locking member 116, preventing distal translation of drive member 110, thereby preventing actuation of the surgical stapling instrument.

Because the staple cartridge is spent and there is no proximally-positioned shuttle to support locking member 116 out of engagement with slot 180, any attempt to re-fire the surgical stapling instrument will be prevented by drive member 110 engaging locking member 116. The only way to again actuate the surgical stapling instrument is to install a fresh, unfired reload.

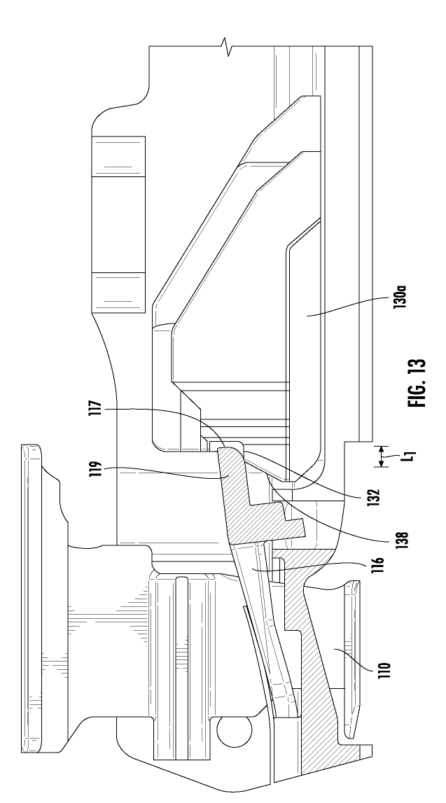

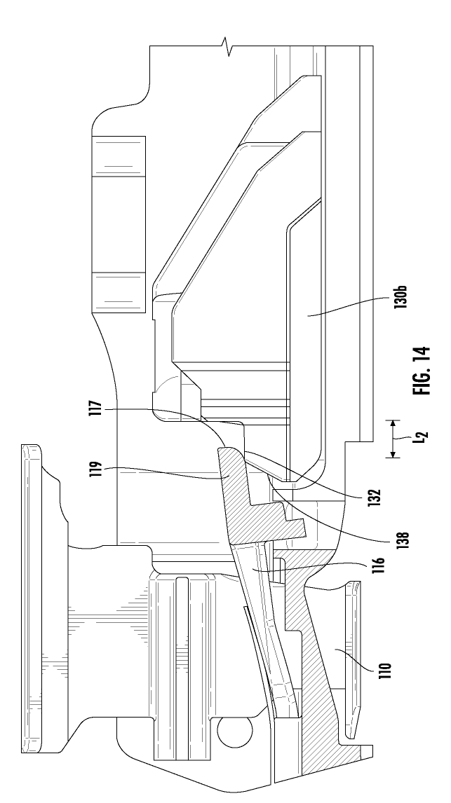

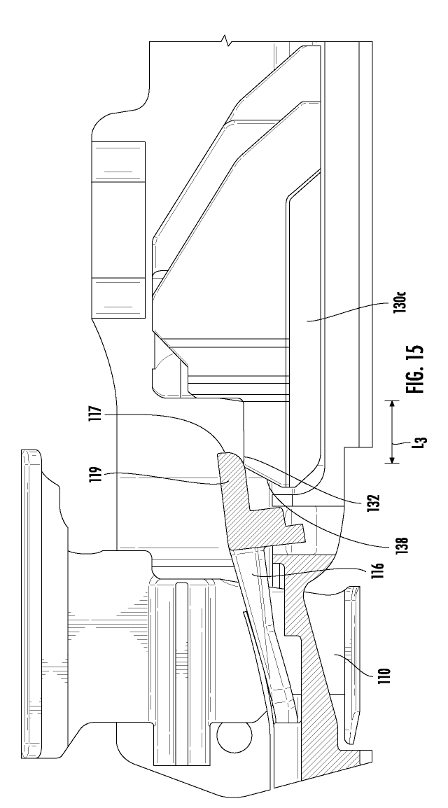

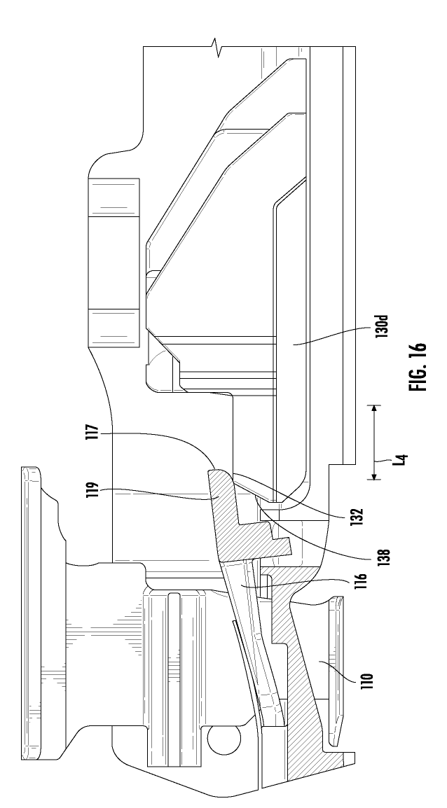

Surgical stapling instruments in accordance with embodiments of this disclosure may be configured to accept and utilize a number of different types of reloads where the different types of reloads have various sizes and arrangements of staples. In embodiments, different reloads may contain reload-specific shuttles 130a-130d having shelves 132 of various lengths (L1-L4) to accommodate the variations in staple configurations. As shown in FIGS. 13-16, these different reloads, despite having different length shelves on the shuttle, all contact the locking member at the same position and angle to urge the locking member onto the shelf. In embodiments, these reloads may be color-coded to indicate the staple configuration and may be utilized in conjunction with a robotics system capable of detecting the type of reload based upon the length of the shelf of the shuttle contained in a given reload.

When each type of reload or cartridge 122 is installed, a proximal ramped edge 138 of shuttle 130 engages a ramped surface 117 on distal portion 119 of locking member 116. The proximal movement of cartridge 122 during installation causes proximal ramped edge 138 of shuttle 130 to slide along ramped surface 117 of distal portion 119 of locking member 116 so that distal portion 119 moves up and onto shelf 132 of shuttle 130, thereby placing the locking member 116 in the first, unlocked position allowing the re-loaded surgical stapling instrument to be actuated and drive member 110 to translate distally through a staple firing stroke as described above.

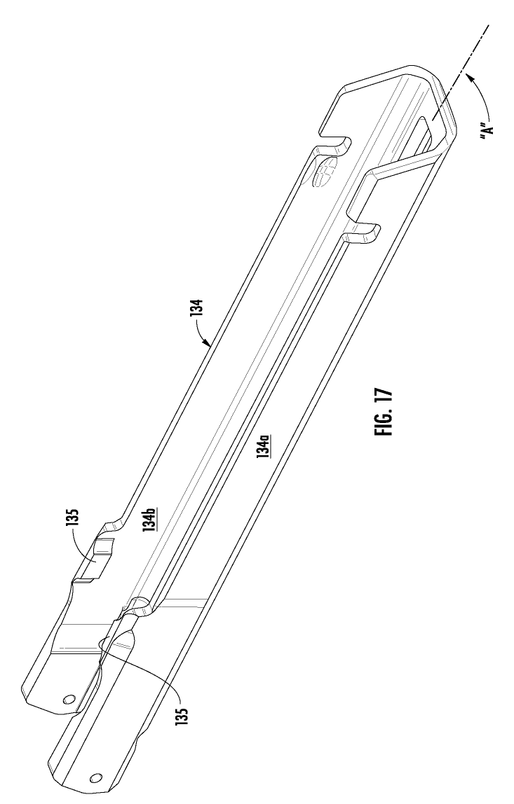

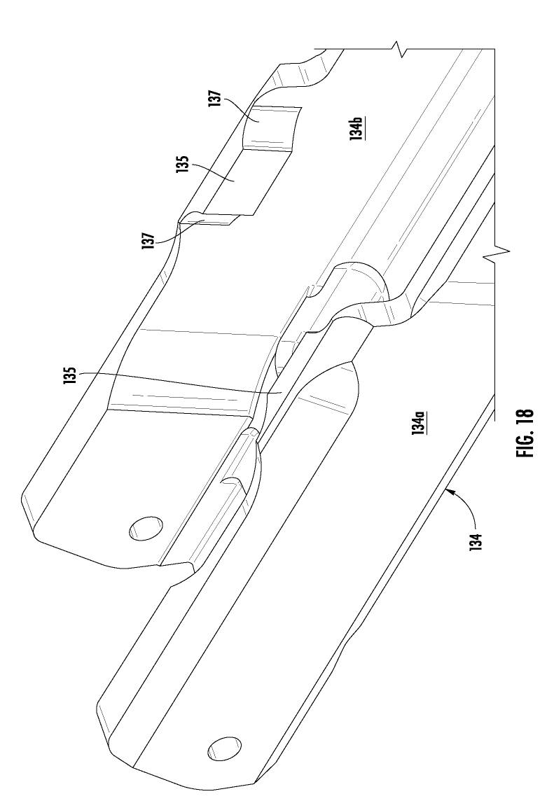

FIG. 17 illustrates a feature for ensuring proper alignment when installing an unfired cartridge (also referred to as a fresh reload). In embodiments, guide structures 135 are formed on sidewalls 134a,b of channel 134 at a substantially proximal position on channel 134. As best seen in FIG. 18, guide structures 135 may contain ramped portions 137 extending in both the proximal and distal directions.

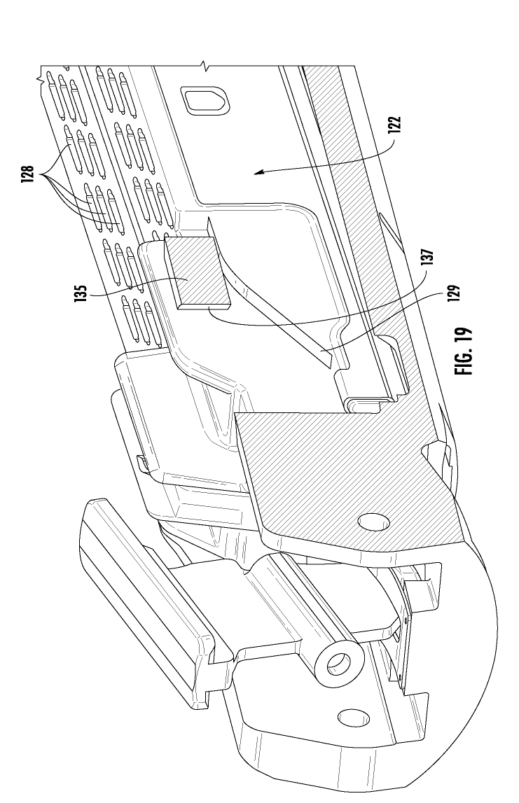

FIG. 19 illustrates an unfired, fresh reload being installed into an illustrative embodiment of a surgical stapling instrument in accordance with this disclosure. Upon installation of the reload, a ramp 129 on cartridge 122 slides underneath guide structure 135, facilitating proper alignment upon reloading. It may be appreciated by those of skill in the art that ramped portions 137 of guide structure 135 help to prevent any edges or corners of cartridge 122 from unintentionally catching on the guide structure. The role of guide structure 135 in properly aligning the reload (and hence shuttle contained therein) assists in guiding ramped edge 138 of shuttle 130 into contact with ramped surface 117 of distal portion 119 of locking member 116 so that locking member 116 rides up proximal ramped portion 138 of shuttle 130 and onto shelf 132 as described above.

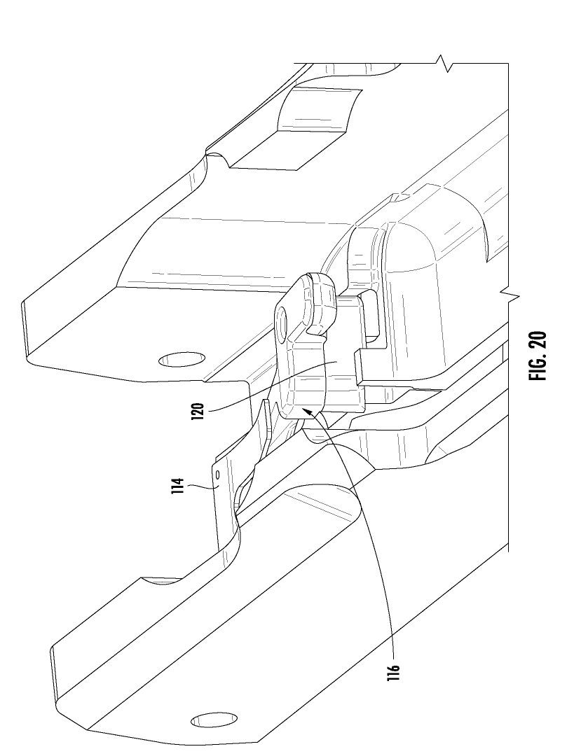

FIG. 20 shows a front wall 120 of locking member 116. Front wall 120 is positioned towards the distal end of locking member 116, as it provides a barrier to protect the rest of locking member 116. It is envisioned that front wall 120 of locking member 116 may prevent a user from accidentally damaging or popping out locking member 116 during cleaning or repair of a surgical stapling instrument in accordance with this disclosure.

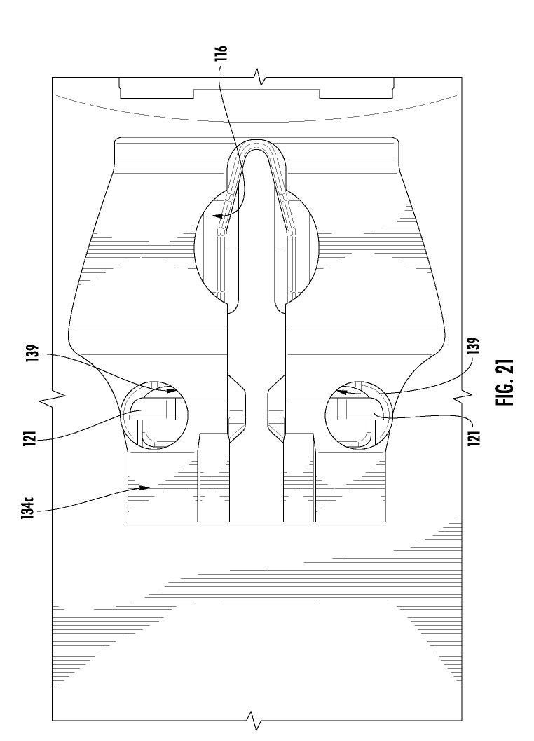

FIG. 21 shows an additional feature of locking member 116. Specifically, FIG. 21 shows a pair of feet 121 configured to pass through openings 139 formed on the bottom wall 134c of channel 134. Feet 121 prevent a user from accidentally removing or repositioning locking member 116. In order to remove locking member 116, a user pinches the pair of feet 121 towards the center of channel 134, and then guide feet 121 up and out through openings 139 of channel 134. Feet 121 allow a user to remove locking member 116 for cleaning if necessary, while also preventing unintentional movement of the locking member when loading, using, or moving a surgical stapling instrument.

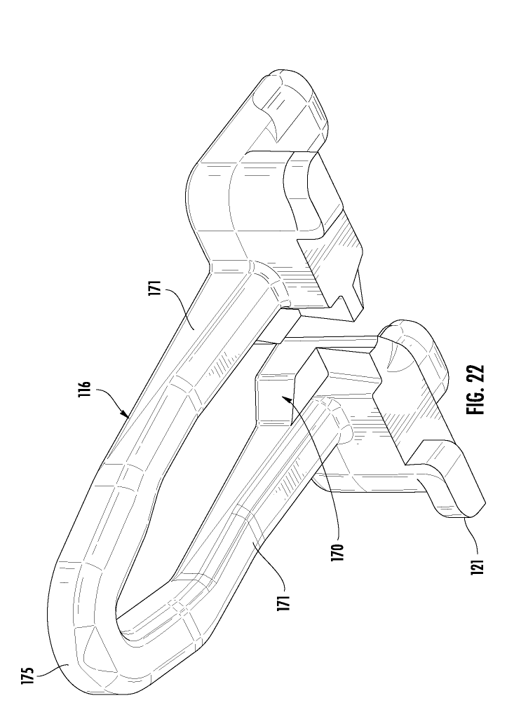

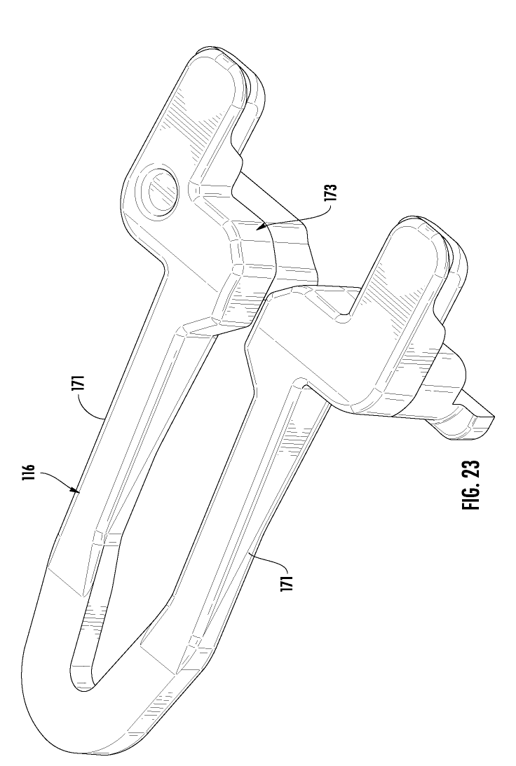

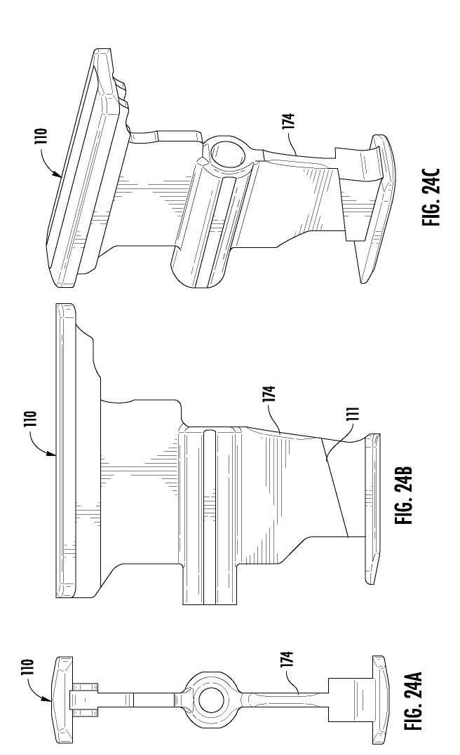

FIG. 22 shows an illustrative embodiment of locking member 116 in accordance with this disclosure having proximally-facing chamfered surfaces 170 formed on distally extending arms 171 of locking member 116. Locking member 116 may be generally “U-Shaped” with distally extending arms 171 originating for a proximal central portion 175 of locking member 116. Proximally-facing chamfered surfaces 170 are configured to engage and align drive member 110 along the longitudinal axis “A” of channel 134 (See FIG. 17) as drive member 110 translates distally between arms 171 of locking member 116. As best seen in FIG. 23, in embodiments locking member 116 may also have distally-facing chamfered surfaces 173 configured to engage and align drive member 110 along the longitudinal axis of channel 134 as drive member 110 retracts and moves between arms 171 of locking member 116. Chamfered surfaces 170 and 173 together help to prevent the drive member 110 from unintentionally contacting any edges or corners of locking member 116, while also promoting proper alignment during translation of drive member 110. As best seen in FIG. 24, drive member 110 may have a chamfered edge 174 to assist in alignment by surfaces 170 and 173.

While several embodiments of this disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As

well, one skilled in the art will appreciate further features and advantages of the present disclosure based on the above-described embodiments. Accordingly, the present disclosure is not to be limited by what has been particularly shown and described, except as indicated by the appended claims.

What is claimed is:

1. A surgical stapling instrument comprising:
a shaft having a longitudinal axis;
an end effector with a first jaw and a second jaw, at least one of the first jaw and the second jaw comprising a longitudinal channel;
a drive member configured to translate in a distal direction through the channel;
a lockout member within one of the first jaw or the second jaw and comprising a first interface portion and a second interface portion spaced from the first interface portion to define a gap therebetween within the longitudinal channel, wherein the lockout member is movable between a first position, permitting distal translation of the drive member, and a second position, wherein the first and second interface portions inhibit distal translation of the drive member.

2. The surgical stapling instrument of claim 1, wherein the lockout member is pivotable about an axis substantially perpendicular to the longitudinal axis.

3. The surgical stapling instrument of claim 1, wherein the first and second interface portions of the lockout member extend at least partially into the longitudinal channel in the first and second positions.

4. The surgical stapling instrument of claim 1, wherein the lockout member is biased into the second position.

5. The surgical stapling instrument of claim 1, further comprising a shuttle configured to translate distally from a proximal position to a distal position within the end effector, wherein the shuttle is configured to maintain the lockout member in the first position when the shuttle is in the proximal position, and to release the lockout member as the shuttle is translated distally to the distal position.

6. The surgical stapling instrument of claim 5, wherein the shuttle comprises a proximal surface, wherein at least a portion of the lockout member engages the proximal surface when the shuttle is in the proximal position.

7. The surgical stapling instrument of claim 5, further comprising a staple cartridge removably coupled to one of the first jaw or the second jaw, wherein the shuttle is disposed within the staple cartridge.

8. The surgical stapling instrument of claim 1, wherein the drive member comprises a central portion and a lateral portion, wherein the first and second interface portions of the lockout member contact the lateral portion of the drive member as the drive member translates in the distal direction when the lockout member is in the second position to block the drive member from moving in the distal direction.

9. The surgical stapling instrument of claim 8, wherein the first and second interface portions of the lockout member are aligned with the central portion of the drive member as the drive member translates in the distal direction when the lockout member is in the first position.

10. The surgical instrument of claim 9, wherein the central portion of the drive member passes through the gap between the first and second interface portions of the lockout member as the drive member is translated in the distal direction.

11. A surgical stapling instrument comprising:
a shaft having a longitudinal axis;
an end effector with a first jaw and a second jaw, at least one of the first jaw and the second jaw comprising a channel;
a drive member configured to translate in a distal direction through the channel, the drive member comprising a central portion and a lateral portion; and
a lockout member within one of the first jaw or the second jaw, wherein the lockout member is movable between a first position, permitting distal translation of the drive member, and a second position, wherein the lockout member contacts the lateral portion of the drive member to prevent distal translation of the drive member.

12. The surgical stapling instrument of claim 11, wherein the lateral portion of the drive member has a first width in a direction perpendicular to the longitudinal channel and the central portion of the drive member has a second width in said direction less than the first width.

13. The surgical instrument of claim 11, wherein the lockout member permits proximal translation of the drive member in the second position.

14. The surgical instrument of claim 13, wherein the lateral portion of the drive member comprises a ramp extending towards the central portion of the drive member in the distal direction.

15. The surgical instrument of claim 14, wherein the lockout member moves along the ramp from the second position to the first position as the drive member is translated in the proximal direction.

16. The surgical stapling instrument of claim 11, wherein the lockout member comprises a first interface portion and a second interface portion spaced from the first interface portion to define a gap therebetween within the longitudinal channel.

17. The surgical stapling instrument of claim 16, wherein the lateral portion of the drive member comprises a first ramped portion and a second ramped portion spaced from the first ramped portion, wherein the first and second interface portions of the lockout member move along the first and second ramped portions of the drive member as the drive member is translated proximally through the end effector.

18. The surgical stapling instrument of claim 11, further comprising a shuttle configured to translate distally from a proximal position to a distal position within the end effector, wherein the shuttle is configured to maintain the lockout member in the first position when the shuttle is in the proximal position, and to release the lockout member as the shuttle is translated distally to the distal position.

19. The surgical stapling instrument of claim 18, wherein the shuttle comprises a proximal surface, wherein at least a portion of the lockout member engages the proximal surface when the shuttle is in the proximal position.

20. The surgical stapling instrument of claim 18, further comprising a staple cartridge removably coupled to one of the first jaw or the second jaw, wherein the shuttle is disposed within the staple cartridge.
